(19) United States
(12) Patent Application Publication
(10) Pub. No.: US 2012/0058110 A1
(43) Pub. Date: Mar. 8, 2012

(54) Method for Predicting the Responsiveness to an Anti-TNF Alpha Antibody Treatment

(75) Inventor: Yannick Jacques, Nantes Cedex (FR)

(73) Assignee: INSERM (INSTITUT NATIONAL DE LA SANTE ET DE LA RECHERCHE MEDICALE), Paris (FR)

(21) Appl. No.: 13/255,291

(22) PCT Filed: Mar. 10, 2009
(86) PCT No.: PCT/IB2009/052732

§ 371 Date: Nov. 18, 2011

(51) Int. Cl. A61K 39/395; A61P 29/00; A61P 17/06; G01N 33/577; A61P 1/00

(52) U.S. Cl. 424/133.1; 424/158.1; 436/501; 424/142.1

(57) ABSTRACT

The present invention relates to a method for predicting the responsiveness of a patient to an anti-TNFα antibody treatment.

### FIELD OF THE INVENTION

The present invention relates to a method for predicting the responsiveness of a patient to an anti-TNFα antibody treatment.

### BACKGROUND OF THE INVENTION

Therapies targeted against cytokines are commonly used in inflammatory bowel disease (IBD). Among them, anti-TNFα monoclonal antibodies are effective and used in clinical routine. Infliximab (IFX) is an anti-TNFα monoclonal antibody which binds soluble and cell-surface TNFα with high affinity and specificity. However, despite its efficacy for the treatment of active Crohn's disease (CD), its anti-inflammatory action is not fully understood.

Anti-TNFα agents could reduce inflammation by neutralizing soluble TNFα and its subsequent interaction with its receptors. However, differences in efficacy profiles despite similar affinities for soluble TNFα suggest that neutralization of soluble TNFα may not be the major mechanism of action. Interestingly, IFX binds to trans-membrane TNFα (tmTNF), and thereby acts either as an antagonist by blocking tmTNF interaction with its receptors or as an agonist by initiating tmTNF mediated reverse signaling leading to cell activation or cytokine suppression (Eissner et al. Cytokine Growth Factor Rev 2004; 15:353-66). Among the molecular partners involved in reverse signaling are the proteases SPPL2a and SPPL2b that participate to tmTNF-mediated induction of interleukin (IL)-12 production (Friedmann et al. Nat Cell Biol 2006; 8:843-8). Accordingly, binding of anti-TNF agents to tmTNF could induce the activation of proteases implicated in the secretion of cytokines or in the cleavage of membrane-anchored receptors. Although several studies had demonstrated that all anti-TNFα bind to tmTNF, there are evidences for differential induction of cytokine suppression through reverse signaling (Kirchner et al. Cytokine 2004; 28:67-74; Mitoma et al. Gastroenterology 2005; 128:376-92; Nesbitt et al Inflamm Bowel Dis 2007; 13:1323-32). For example, it was demonstrated that IFX but not etanercept could down-regulate IL-1α and TNFα secretions and increase IL-10 secretion. These differences could explain the unequal efficacy of these agents and in particular the poor efficacy of etanercept in CD.

Among pro-inflammatory cytokines, IL-15 plays a key role in the activation of innate and adaptative immune responses and its modulation by anti-TNF agents could deeply regulate the inflammatory process in CD. IL-15 was identified for its IL-2 like ability to stimulate the growth of T cells but experiments have shown that the two cytokines exert complementary effects within the immune system. IL-15 is produced by macrophages and also by non-hematopoietic cell types such as enterocytes.

IL-15 binds a trimolecular IL-15 receptor (IL-15R) complex formed by the β and γ chains of the IL-2R and a specific receptor chain (IL-15Rα). Soluble forms of IL-15Rα retains a high affinity for IL-15 and can act either as a powerful antagonist of IL-15 action or as an agonist of IL-15 action through the IL-15Rβ/γ complex. Natural shedding of such soluble IL-15Rα has been shown to result from the cleavage of tmIL-15Rα by metalloproteinase-dependent proteolytic mechanisms involving ADAM17 also known as TACE (TNF-α converting enzyme).

The role of IL-15 and its soluble receptor in IBD is not clearly understood. IL-15 expression has been shown in intestinal epithelial cells as well as in the intestinal mucosa. Moreover, IL-15 positive cells are more numerous in the lamina propria of CD and UC patients compared to uninflamed control patients. Neutralization of IL-15 had various effects in animal models of IBD. It was shown that IL-15 neutralization by sIL-15Rα could aggravate epithelial damage and increase inflammation in the DSS-induced colitis mice model, whereas IL-15 neutralization reduced inflammation and the mucosal cell infiltrate in the SCID transfer model.

### SUMMARY OF THE INVENTION

The present invention relates to a method for predicting the responsiveness of a patient to an anti-TNFα antibody treatment, said method comprising measuring the level of IL-15 in a biological sample obtained from said patient.

A high level of IL-15 is predictive of a response to an anti-TNFα antibody treatment.

The present invention also relates to a method for treating a patient, comprising the step of administering an effective amount of an anti-TNFα antibody to said patient, wherein said patient has a high level of IL-15.

The present invention relates to an anti-TNFα antibody for the treatment of a patient, wherein said patient has a high level of IL-15.

### DETAILED DESCRIPTION OF THE INVENTION

The present invention relates to a method for predicting the responsiveness of a patient to an anti-TNFα antibody treatment, said method comprising the step of measuring the level of IL-15 in a biological sample obtained from said patient.

A high level of IL-15 is predictive of a response to an anti-TNFα antibody treatment. Typically, the method of the invention may comprise a step of comparing the level of IL-15 with a cut-off value. Typically, the cut-off value may be the IL-15 level of healthy subjects. Typically, said cut-off value may be determined by receiver operating characteristic curve analysis.

The present invention relates to a method for determining if a patient suffering from an inflammatory disease is responsive to an anti-TNFα antibody treatment comprising the step of measuring the level of IL-15 in a biological sample obtained from said patient.

The present invention also provides a method which combines the selection of a specific population of patients that are likely to be responsive to treatment regimen with an anti-TNFα antibody, with the treatment with an anti-TNFα antibody of said population of patients.

Also provided is an anti-TNFα antibody for the treatment of a patient suffering from an inflammatory disease, wherein said patient has a high level of IL-15.

Typically a patient who can be treated with anti-TNFα antibody is a patient suffering from an inflammatory disease.

Examples of inflammatory diseases which can be treated with an anti-TNFα antibody are inflammatory bowel diseases, typically Crohn's disease, rheumatoid arthritis, ankylosing spondylitis and psoriasis.

Examples of anti-TNFα antibodies are infliximab (IFX) (a mouse-human chimeric whole antibody) (Remicade™; Centocor, Horsham, Pa., USA), adalimumab (a recombinant human whole antibody) (Humira; Abbott Laboratories, Abbott Park, Ill., USA) and Certolizumab pegol (Cimzia™; UCB, Belgium), which is a PEGylated Fab′ fragment of a humanized monoclonal antibody that binds and neutralizes human TNFα.

In a preferred embodiment said anti-TNFα antibody is infliximab.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments or derivatives. Antibody fragments include but are not limited to Fv, Fab, F(ab′)₂, Fab′, dsFv, scFv, sc(Fv)₂ and diabodies.

The terms “biological sample” as used herein refer to a biological sample obtained for the purpose of in vitro evaluation. In the methods of the present invention, the sample or patient sample preferably may comprise any body fluid. Typical biological samples to be used in the method according to the invention are blood samples (e.g. whole blood sample, serum sample, or plasma sample). In a preferred embodiment said blood sample is a serum sample.

Once the biological sample from the patient is prepared, the level of IL-15 or a fragment thereof may be measured by any known method in the art.

For example, the concentration of IL-15 or a fragment thereof may be measured by using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, high performance liquid chromatography (HPLC), size exclusion chromatography, solid-phase affinity, etc.

In a particular embodiment, such methods comprise contacting the biological sample with a binding partner capable of selectively interacting with IL-15 or a fragment thereof present in the biological sample.

The binding partner may be generally an antibody that may be polyclonal or monoclonal, preferably monoclonal. Polyclonal antibodies directed against IL-15 or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against IL-15 can be prepared and isolated using any technique that provides for the production of antibody molecules by continuous cell lines in culture. Techniques for production and isolation include but are not limited to the hybridoma technique originally described by Kohler et al. Nature. 1975; 256(5517):495-7; the human B-cell hybridoma technique (Cote et al Proc Natl Acad Sci USA. 1983; 80(7):2026-30); and the EBV-hybridoma technique (Cole et al., 1985, In Monoclonal Antibodies and Cancer Therapy (Alan Liss, Inc.) pp. 77-96). Alternatively, techniques described for the production of single chain antibodies (see e.g. U.S. Pat. No. 4,946,778) can be adapted to produce anti-CGA, single chain antibodies. Antibodies useful in practicing the present invention also include anti-IL-15 fragments including but not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab and/or scFv expression libraries can be constructed to allow rapid identification of fragments having the desired specificity to IL-15. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e.g., bacteria, the phage displays the antibody fragment. Phage display of antibodies may also be provided by combinatorial methods known to those skilled in the art. Antibody fragments displayed by a phage may then be used as part of an immunoassay.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk et al. (1990) Science, 249, 505-510. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena (1999) Clin Chem. 45(9):1628-50. Peptide aptamers consist of conformationally constrained antibody variable regions displayed by a platform protein, such as E. coli Thioredoxin A, that are selected from combinatorial libraries by two hybrid methods (Colas et al. (1996). Nature, 380, 548-50).

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal.

As used herein, the term “labeled”, with regard to the antibody, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example radioactive molecules include but are not limited radioactive atom for scintigraphic studies such as I123, I124, In111, Re186, Re188.

The aforementioned assays generally involve the bounding of the binding partner (ie. Antibody or aptamer) in a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against IL-15 or a fragment thereof. A biological sample containing or suspected of containing IL-15 or a fragment thereof is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Different immunoassays, such as radioimmunoassay or ELISA have been described in the art.

Measuring the concentration of IL-15 (with or without immunoassay-based methods) may also include separation of the proteins: centrifugation based on the protein's molecular weight; electrophoresis based on mass and charge; HPLC based on hydrophobicity; size exclusion chromatography based on size; and solid-phase affinity based on the protein's affinity for the particular solid-phase that is use. Once separated, IL-15 may be identified based on the known “separation profile” e.g., retention time, for that protein and measured using standard techniques. Alternatively, the separated proteins may be detected and measured by, for example, a mass spectrometer.

The invention will further be illustrated in view of the following figures and example.

### FIGURE LEGENDS

FIG. 1: Clinical and biological markers of activity. The CDAI scores were calculated before and after IFX in CD patients. Total TNF and CRP levels were measured in the sera of 43 CD patients before and after 3 infusions of IFX and in 37 healthy controls. In responder patients (A, B, C panels), CDAI scores (A), TNF (B) and CRP (C) levels were significantly higher compared to controls and decreased after 3 infusions of IFX. To the opposite, in non-responder patients (D, E, F panels), the CDAI scores (D), TNF levels (E) and CRP (F) which were significantly higher before the treatment compared to controls remained elevated after the treatment. *** P<0.001.

FIG. 2: IL-15 levels in the serum of CD patients before and after 3 infusions of IFX. In responder patients (A), IL-15 levels were significantly higher than in healthy controls before IFX and decreased significantly after 3 infusions of IFX. To the opposite, in non-responder patients (B), IL-15 levels were comparable to those of the controls and not modified after IFX. * P<0.05

FIG. 3: sIL-15Rα levels in the serum of CD patients before and after 3 infusions of IFX. In responder patients (A), sIL-15Rα levels were significantly higher than in healthy controls before IFX and increased significantly after 3 infusions of IFX. To the opposite, in non-responder patients (B), sIL-15Rα levels were comparable to those of the controls and not modified after IFX. * P<0.05

FIG. 4: sIL-15Rα secretion by human macrophages isolated from peripheral blood samples of healthy donors. Macrophages were cultured in medium supplemented with IFX or etanercept (A) with or without (for IFX) an inhibitor of metalloprotease (GM-6001) (B). sIL-15Rα secretion increased significantly when macrophages were cultured in the presence of IFX compared to the cells cultured in absence of IFX (2.0±0.6 versus 18.0±1.2 pM). sIL-15Rα secretion was significantly lower when macrophages were treated by etanercept compared to macrophages cultured in the presence of IFX (6.3±0.9 versus 18.0±1.2 pM).

The sIL-15Rα secretion was significantly lower when macrophages were co-treated by IFX and GM-6001 compared to macrophages cultured in the presence of IFX alone (3.8±0.8 versus 22.4±2.0 pM). sIL-15Rα secretion was not modified when macrophages were cultured in the presence of GM-6001 compared to control cells (1.4±0.9 versus 4.5±3.0 pM). ** P<0.005; * P<0.05

TABLE
Table 1: clinical and demographic characteristics of the patients at
baseline.

| | |
|---|---|
| Patients (n) | 40 |
| Sex ratio (male/female) | 12/28 |
| Mean age - years (SD) | 37.8 (14.1) |
| Mean duration of the disease - years (SD) | 8.7 (7.4) |
| Tobacco use (%) | 52 |
| Location of the disease - n (%) | |
| Ileum | 2 (5.0) |
| Colonic | 25 (62.5) |
| Ileo-colonic | 13 (32.5) |
| Perineal^{a} | 11 (27.5) |
| Type of the disease - n (%) | |
| Non-stricturing and non-penetrating | 34 (85) |
| Stricturing | 6 (15) |
| penetrating | 0 (0) |
| Resection - n (%) | 12 (30) |
| Treatment - n (%) | |
| Corticosteroids alone | 3 (7.5) |
| Immunosuppressors alone | 30 (75.0) |
| Corticosteroids and immunosuppressors | 6 (15) |

^{a}perineal disease is not exclusive and can be associated with other locations.

### Example

### Abstract

We investigated serum levels and cellular expression of IL-15 and sIL-15Rα in Crohn's disease (CD) patients during treatment by infliximab (IFX), and the effect of IFX on sIL-15Rα secretion by macrophages.

Forty patients were studied, 35 received a single IFX course (3 infusions) while 5 were treated twice. 37 healthy controls were also included. Serum levels of IL-15 and sIL-15Rα were determined by RIA and TNFα by bio-assay. Cellular expressions of IL-15Rα and ADAM17 were assessed by immunohistochemistry (IHC). For in vitro studies, human macrophages were cultured with IFX or etanercept (inefficient in CD) with or without GM-6001 an inhibitor of metalloproteinases.

35 and 10 patients were classified as responders and non-responders to IFX according to the TNF, and CRP levels and CDAI. Before IFX, IL-15 was significantly higher in responders than in controls and non-responders. After IFX, IL-15 decreased in responders while remaining stable in non-responders. sIL-15Rα levels were also higher in CD than in controls and increased only in responders after IFX. IL-15Rα and ADAM17 cellular expressions co-localized in epithelial cells and monocytes and were higher in CD compared to controls. In vitro, IFX but not etanercept induced sIL-15Rα secretion by activated macrophages, an effect inhibited by GM-6001.

IL-15 and sIL-15Rα are increased in patients that respond to IFX treatment and the response is associated with a decrease of IL-15 and an increase of sIL-15Rα. IFX but not etanercept induced the cleavage of the IL-15 receptor by a metalloproteinase, suggesting a specific modulation of IL-15 and its soluble receptor by reverse signaling through tmTNF.

### Patients and Methods

### Patients

Patients with active CD were treated by 3 infusions of IFX (5 mg/Kg) at week 0, 2 and 6. Thirty-five patients received one IFX course while 5 others were treated twice. A clinical response to IFX was mostly defined by a Crohn's Disease Activity Index (CDAI) under 150 points or by the physician global judgment in 6 patients. Clinical response for patients treated for perineal disease was assessed by the disappearance of draining fistulas. Blood samples were taken before each infusion and were centrifuged and stored at −80° C. Blood samples were also collected in 37 healthy volunteers. Forty patients with active CD were included in the study (12 males, 28 females, mean age 37.8 years). According to the Montreal classification, the disease was predominantly located into the colon (L2) or the ileocolon (L3) in 62.5 and 32.5% respectively and the disease was mostly non-stricturing and non-penetrating (B1). Only 11 patients (27.5%) had a perineal disease (p), 7 were treated for fistulas alone, the others for a combination of fistulas and a non-stricturing and non-penetrating disease (B1, p). Six patients were treated by a combination of steroids and immunosuppressors, 3 patients received only steroids, 30 only immunosuppressors. Treatments were not modified except steroids which were always stopped between the first and the third infusion. Clinical and demographic data are summarized in table 1. There were no clinical and demographic differences between patients who experienced a response or not to IFX.

Radioimmunoassay for Quantification of sIL-15Rα and IL-15.

The quantification of sIL-15Rα and IL-15 was determined as previously described (Mortier et al. J Immunol 2004; 173:1681-8). Sandwich RIAs were set up in which the goat anti-human IL-15Rα Ab AF247 or the mouse anti-human IL-15 Mab247 (R&D) was used as capture Ab and radio-iodinated anti-human IL-15Rα Ab M161 (GenMab; Copenhagen, Denmark) or radio-iodinated mouse anti human IL-15 BE-29 (Diaclone: Besançon, France) was used as tracer. A purified recombinant sIL-15Rα protein (Bernard et al. J Biol Chem 2004; 279:24313-22) and recombinant IL-15 (Peprotech) were used as standards. Capture Abs were coated (5 μg/mL; 50 μL/well) to high-adsorption Nunc maxisorp plates (Fisher Bioblock Scientific, Illkirch, France). Wells were saturated with PBS-BSA 0.5% for 15 min and samples (50 μL/well) were incubated for 1 h at 4° C. The M161 or BE-29 mAbs were iodinated using the iodogen method and then added (1 nM, 50 μL/well) for 1 h at 4° C. Supernatants of each well were collected and the wells washed twice with PBS. The radioactivity associated to the wells (bound fraction) and contained in the supernatants and washes (unbound fraction) were determined.

The upper normal value of IL-15 (6.1 pM) was calculated by the mean+2 SD of the values measured in healthy volunteers.

### Quantification Assay of TNFα

TNFα concentrations were measured in the serum of 43 patients (33 responder patients, 10 non-responders). Quantification TNFα was performed by the XTT method using the murine cell line WEHI 164 (ATCC N^{o} CRL1751) (Espevik et al. J Immunol Methods 1986; 95:99-105). Cells were seeded in 96-well plates (3×10⁴ cells/50 μL/well) and grown at 37° C. in RPMI 1640 supplemented with 0.1 nmol/L of glutamine, 10% heat-inactivated FCS, LiCl and actinomycin D. Sera from CD patients and healthy volunteers were serially diluted, added to wells and a purified recombinant TNFα (R&D) was used as standard. After 20 h of culture, 50 μL/well of XTT and electron coupling reagent was added and cell survival was determined by measuring the optical density at 450 nm. The sensitivity of the assay was 0.10 pg/mL. The TNFα upper normal limit was calculated by the mean+2 SD of the values obtained in the healthy volunteers.

### Isolation of Human Macrophages

Human monocytes were isolated from blood samples of healthy donors. PBMC were collected after Ficoll-Hypaque density centrifugation, resuspended at 7×10⁶ cells/mL and allowed to adhere in culture flasks (RPMI 1640 supplemented with L-glutamine, streptomycin, penicillin, 10% FCS) for 1 h 30 at 37° C. Nonadherent cells were removed and adherent cells were washed five times with PBS. Monocytes were further differentiated for 5 days in the same medium supplemented with GM-CSF (100 ng/mL). For induction, monocytes were seeded in multiwell plates at 10⁴ cells/well in 100 μL and cultured for 8 h in medium supplemented with a fixed concentration (0.01 μg/mL) of IFX, etanercept or GM-SCF. In the case of IFX, a broad spectrum inhibitor of metalloproteases (GM-6001) was tested at a concentration of 0.2 nM. After incubation, supernatants were obtained by centrifugation.

### Immuno-Fluorescence Staining

Colonic biopsies were obtained from three CD patients before the first and the third IFX infusion and two healthy control patients. All patients had clinical responses and a mucosal healing in two patients. Colonic biopsies were immediately frozen and stored at −80° C. Specimens were sectioned at 4 to 6 μm with a cryostat, placed on slides, air dried, and fixed for 10 min with 100% acetone. Before incubation with primary antibodies, the slides were saturated with PBS/BSA 0.5%. Primary antibodies were goat anti-IL-15Rα and rabbit anti-ADAM17 (R&D, Abingdom, UK) used at concentrations of 15 μg/mL and 10 μg/mL respectively. Secondary antibodies were rabbit anti-goat IgG Alexa 488 and chicken anti-rabbit IgG FITC used at a concentration of 2 μg/mL. Isotype-matched antibodies were used as negative controls. In the double immunofluorescence experiments, we checked that each secondary antibody did not cross-react with the primary antibody of the other immunoglobulin species. Fluorescent images were analyzed with an epifluorescent microscope DMR (Leica Microsystems).

### Statistical Analysis

Quantitative data were expressed as mean±standard deviation (SD) or median (IQ 25-75). The normality of the distribution was analyzed by the Kolmogorov-Smirnov test. Differences between quantitative data were assessed by the paired or unpaired t-test and the Mann-Whitney U test or the Wilcoxon signed rank test for non-normally distributed data. Correlations between parameters were determined using Spearman's rank correlation test. The Fischer exact test was performed to compare the proportion of patient responders and non-responders in whom IL-15, sIL-15Rα and CRP were under the normal limit and, in whom the diagnosis of CD was made under the age of forty and the disease was located to the colon. Receiver operating curves were plotted to determine the accuracy of IL-15 measurement as a predictive factor of no-response to infliximab. A P value of less than 0.05 was considered statistically significant.

### Results

### Clinical and Biological Markers of Activity

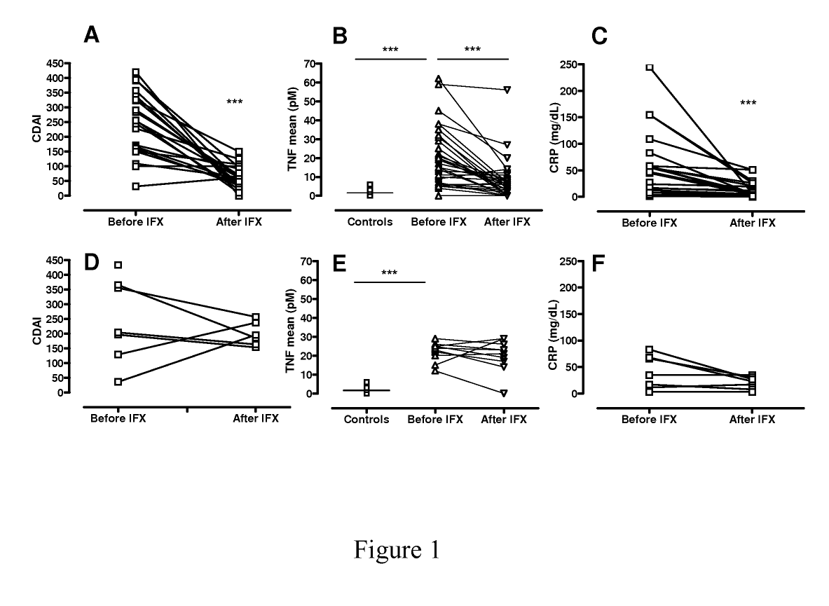

The mean±SD of the CDAI was not different between responder and non-responder patients before IFX (245.1±108.2 versus 245.4±143.2). After IFX, the mean of the CDAI decreased significantly in responder patients (58.9±35.0 versus 245.1±108.2; P<0.001), whereas no difference was found in non-responder patients (198.3±41.1 versus 245.4±143.2; P=0.8) (FIG. 1).

Circulating TNFα was above the normal value in 35 (81%) patients before the first infusion of IFX. Its mean level was significantly higher in non-responder patients (22.1±5.2 pM) and in responder patients (20.3±15.8 pM) than in control (1.6±2.3 pM) (P<0.001) but no significant difference was found between the two groups of patients (FIG. 1). At week 6, TNFα decreased in 94% (31/33) of responder patients with individual values dropping to control ranges in 61% of patients (20/33). At the opposite, TNF remained above the control value in 90% (9/10) of non-responder patients. The mean of TNF levels significantly decreased in responder patients (7.3±10.8 versus 20.3±15.8 pM; P<0.001) whereas no difference was found in non-responder patients (22.1±5.2 versus 20.1±8.6).

The mean of the CRP levels was not significantly different between responder and non-responder patients before IFX (32.2±32.0 versus 39.3±55.0 mg/dL; P=0.7). After IFX, CRP decreased significantly in responder patients (10.0±13.2 versus 39.3±55.0 mg/dL; P<0.001), whereas no difference was found in non-responder patients (16.7±12.8 versus 32.2±32.0 mg/dL; P=0.2) (FIG. 1).

IL-15, sIL-15Rα in CD Patients Before IFX

IL-15 was detectable in the serum of 40% (4/10) of non-responder patients and in 74% (26/35) of responder patients. On average for the 45 patients before treatment, IL-15 levels (mean±SD) were significantly higher in CD patients than in healthy subjects (8.8±15.2 versus 1.3±2.4 pM; P<0.02). IL-15 levels was also significantly higher in responder than in non-responder patients (11.1±16.7 versus 0.7±1.3; P=0.01) and than in healthy controls (11.1±16.7 versus 1.3±2.4 pM P=0.002). To the opposite, IL-15 levels were not different between non-responder patients and healthy subjects. Soluble IL-15Rα was also detected in the serum of CD patients. sIL-15Rα was significantly higher in CD patients before treatment than in healthy subjects (11.1±20.6 versus 0.4±1.7 pM; P<0.001). Although sIL-15Rα levels were higher in responder than in non-responder patients, the difference was not significant (12.7±23.0 versus 6.0±7.3 pM). Moreover, sIL-15Rα in responder and non-responder patients were both significantly higher than in healthy subject (P<0.001 and P=0.003, respectively).

Effect of IFX on IL-15 and sIL-15-Rα Concentrations

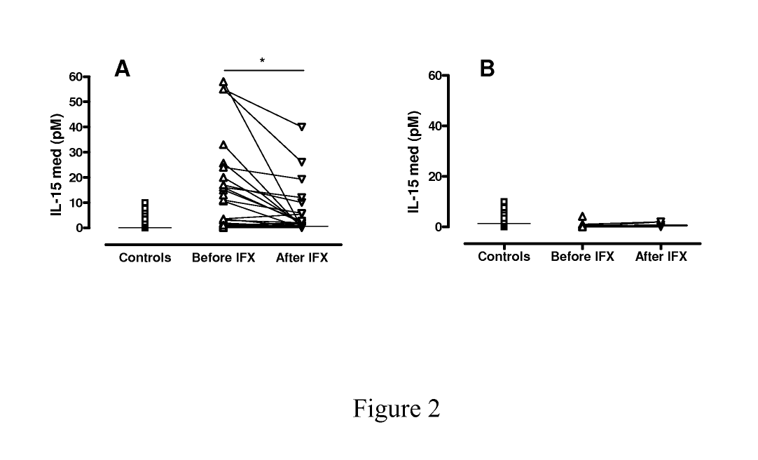

When patients responded to IFX, the median (IQ) of the IL-15 levels significantly decreased from 1.9 pM (0.1-16.0) to 0.7 pM (0.0-2.5) (P=0.02) (FIG. 2). The number of patients in whom IL-15 was undetectable increased from 26% (9/35) before to 43% (15/35) after IFX and IL-15 dropped below the control value in 83% (29/35). To the opposite, in non-responder patients, the median of the IL-15 levels was not modified after IFX (FIG. 2).

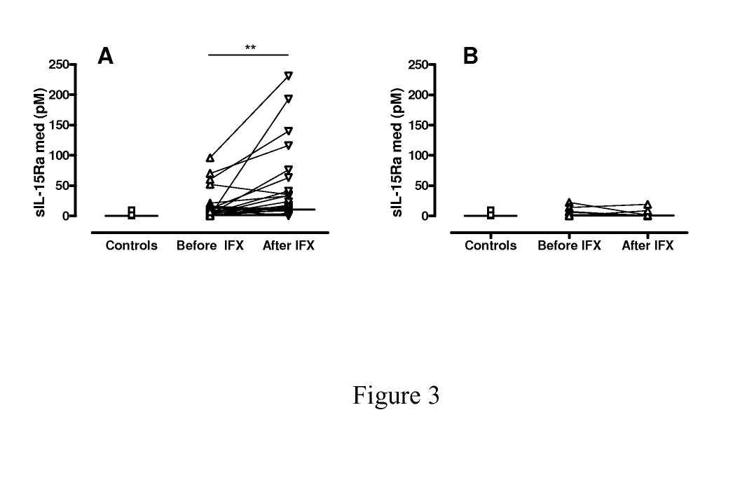

In responder patients, the median (IQ) of the sIL-15Rα levels significantly increased after IFX from 3.0 pM (3.0-13.25) to 10.5 pM (0.6-34.5) (P=0.002) and the proportion of patients with detectable sIL-15Rα increased by 38% after IFX (FIG. 3). To the opposite, in non-responder patients, sIL-15Rα levels did not change significantly (5.99±7.3 versus 3.5±6.4 pM; P=0.3) (FIG. 3).

IFX Induced sIL-15Rα Secretion

Macrophages constitutively secrete low levels of sIL-15Rα. This secretion was markedly increased upon addition of IFX (18.0±1.2 pM versus 2.0±0.6 pM; P<0.001). In contrast, addition of etanercept did not significantly affect sIL-15Rα secretion (6.3±0.9 versus 2.0±0.6 pM; P=0.1).

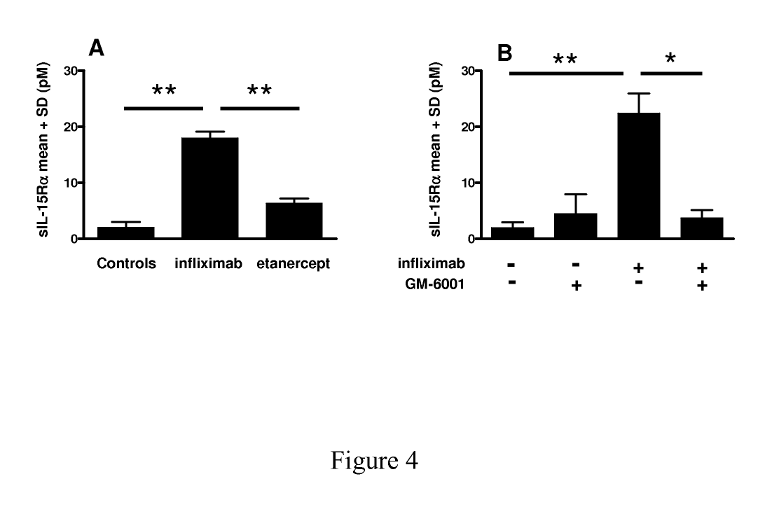

The inducing effect of IFX on sIL-15Rα secretion was completely inhibited by GM-6001. sIL-15Rα secretion measured in the presence of IFX and GM-6001 was significantly lower than that measured in the presence of IFX alone (3.8±0.8 versus 22.4±2.0 pM; P=0.001) and not significantly different from control levels (3.8±0.8 versus 1.4±0.9; P>0.1). The effect of GM-6001 was not due to a direct down-regulation of sIL-15Rα secretion because it did not affect basal sIL-15Rα secretion (1.4±0.9 versus 4.5±3.0 pM; P=0.3) (FIG. 4).

### Cellular Expression of IL-15Rα and the Metalloproteinase ADAM17 in Colonic Mucosa

Immunofluorescence showed that IL-15Rα and ADAM17 were co-localized both in colonic epithelial cells and monocytes in CD patients and in healthy controls (not shown). Before IFX, the cellular expression of ADAM17 and IL-15Rα were higher in CD patients compared to healthy controls. These expressions were not modified after IFX. However, the cellular expression of ADAM17 and IL-15Rα before IFX were higher for the two patients in whom IFX induced a mucosal healing and an histological improvement compared to the patient who had only a clinical response without mucosal healing.

### Predictive Factors of No-Response to Infliximab

The predictors of primary no-response to IFX tested in this study were the age at onset of CD (more than 40 years), the colonic location of the disease at the time of IFX (versus ileum or ileo-colonic location), a concomitant treatment with immunosuppressor, a CRP below the upper normal limit and a serum IL-15 level below the estimated upper limit (6.1 pM). Among these predictors, only a serum IL-15 concentration below the normal value was found associated with a significant risk of no-response to IFX (P=0.002).

We performed receiver operating curve analyses to determine cut-off points for IL-15 levels. A cut-off point of 1.9 pM was used to distinguish response and no-response to IFX and we could determine a sensitivity of 90% and a specificity of 50%.

### Discussion

This study demonstrates for the first time the in vivo expression of a naturally soluble form of the human IL-15Rα in CD and its modulation according to the inflammatory status of the patients. The serum levels of sIL-15Rα were significantly higher in active CD patients than in controls and, after IFX treatment, sIL-15Rα increased in responder patients while it decreased in non-responders. sIL-15Rα secretion was also associated with a secretion of IL-15 which, to the opposite decreased in responder patients after IFX and remained stable in non-responders. The in vitro results suggest that the increase in sIL-15Rα could be due to a direct effect of IFX on cellular tmTNF and not only a consequence of the decreased inflammation. The down-stream mechanisms seemed to be specific of IFX, as etanercept, did not induce the release of sIL-15Rα by activated macrophages.

Our in vivo data demonstrated that IL-15 significantly decreased in patients who experienced a clinical response after IFX. This could be due, in part, to a general benefice on the inflammation, but also could be due to the release of sIL-15Rα from IL-15Rα bearing cells. sIL-15Rα retains a high affinity for IL-15 and could subsequently decrease circulating IL-15 levels by forming IL-15/sIL15-Rα complexes. Indeed, our in vitro data showed that IFX induced the secretion of sIL-15Rα by activated macrophages. This effect was inhibited by an inhibitor of metalloproteases, suggesting that IFX could induce, via its binding on tmTNF, the downstream activation of a metalloproteases leading to the cleavage of tmIL-15Rα. Cleavage of tmIL-15Rα by metalloproteinases is also supported by our IHC data showing that i) IL15-Rα and ADAM17 are co-localized at the level of epithelial cell membranes and ii) both proteins have increased expression levels after IFX. Such IFX-induced release of sIL-15Rα could explain the increase of sIL-15Rα in the sera of responder patients and the parallel decrease of IL-15. To the opposite, in non-responder patients, IFX was unable to modulate sIL-15Rα or IL-15, suggesting a default in the downstream pathways leading to the cleavage of tm IL-15Rα. Among the protein potentially involved in these pathways, ADAM17 cleaves many membrane proteins including tmIL-15Rα. In a genetic analysis of a cohort of CD, it was demonstrated that one haplotype of the ADAM17 gene was associated with a good response to IFX suggesting that the ADAM17 gene is involved in the response to IFX as part of a multigenetic mechanism (Dideberg et al. Pharmacogenet Genomics 2006; 16:727-34). In our patients, no-response to the treatment could be due in part to a default of the ADAM17 activation by IFX preventing the cleavage of the tmIL-15Rα.

In non responder patients, IL-15 levels were comparable to those obtained in controls and lower than in responder patients. One could suggest that our population of non-responder patients included non-inflamed patients with irritable bowel symptoms. This is however very unlikely because the TNF and CRP levels as well as the CDAI scores before IFX were comparable to those exhibited by responder patients, therefore allowing to consider them as real non-responders to IFX. The inefficacy of IFX was not explained by an imbalance between TNF-α and TNF-β as their proportions were similar in the sera of responder and non-responder patients (data not shown). The discrepancy between the IL-15 levels could neither be explained by differences in the demographic, clinical or therapeutic characteristics of the responder versus non-responder patients. One explanation could be linked to the mechanisms involved in cell secretion of IL-15. Indeed, it has recently been shown that IL-15 secretion is highly dependent on its intracellular pre-association with IL-15Rα. IL-15Rα binds tightly to IL-15 and stabilizes the protein in the endoplasmic reticulum/golgi apparatus. The IL-15/IL-15Rα complexes and excess uncomplexed IL-15Rα are translocated to the plasma membrane (Duitman et al. Mol Cell Biol 2008; 28:4851-61; Mortier et al. J Exp Med 2008; 205:1213-25) where they can be cleaved by matrix metalloproteinases, resulting in the shedding of sIL-15Rα and soluble IL-15/sIL-15Rα complexes. The differences in IL-15 concentrations between responder and non-responder patients could be explained by a default either in the mechanisms leading to the translocation of the complex to the membrane or, as it was suggested above, in the cleavage of the tmIL-15Rα by matrix-metalloproteinases. The latter mechanism could also explain the primary low levels of IL-15 and sIL-15Rα observed in non-responder patients even in the presence of sustained intestinal inflammation as assessed by elevated CRP and TNF levels.

Modulation of IL-15 and sIL-15Rα secretions by anti-TNF agents may be of particular interest to control the inflammatory response during IBD. As demonstrated from data obtained in IL-15-transgenic and IL-15 deficient mice, IL-15 plays a significant role in the inflammatory joint destruction in which the inflammatory response is quite similar to CD (McInnes et al. Nat Med 1997; 3:189-95; Yoshihara et al. Eur J Immunol 2007; 37:2744-52). In murine collagen-induced arthritis, IL-15 induced IL-17 production and IL-23R expression in T cells and IL-15 synergized with IL-23 to induce production of IL-17. Similar mechanisms implicating IL-15 over-expression could therefore operate in other inflammatory diseases in which Th17 responses have been described. Moreover, the IFX-induced release of sIL-15Rα could also participate to the restoration of cell apoptosis which was described in patients with active CD (Boirivant et al Gastroenterology 1999; 116:557-65. Di Sabatino et al. Gut 2004; 53:70-7). IL-15 is a well known potent inhibitor of apoptosis by blocking adaptor protein recruitment to the TNF receptor (TNFR1). Intracellular domains of ligand stimulated TNFR1 and tmIL-15Rα compete for binding TRAF2 with tmIL-15Rα showing a higher affinity for TRAF2 than TNFR1. Then, upon activation with IL-15, tmIL-15Rα rapidly depletes TRAF2 which becomes unavailable for assembly with the TNF-TNFR1 complex. We could speculate that in responder patients, the IFX-induced shedding of sIL-15Rα could release TRAF2 and restore its availability for the TNF-TNFR1 complex which in turn induces cell apoptosis.

In conclusion, our results demonstrate that, in CD patients IL-15 and its soluble receptor are implicated in the inflammatory cascade. They also suggest that patients resistant to IFX exhibit a defect in the mechanisms leading to the secretion of the IL-15/sIL-15Rα complex. This defect remains unexplained but could implicate the metallo-proteinase pathways responsible for the shedding of sIL-15Rα. Additionally, no significant clinical, demographic or biological differences could be seen between responder and non-responder patients to IFX, except IL-15 levels. Among the different factors tested, only a low level of IL-15 was predictive of a no-response to IFX.

### REFERENCES

Throughout this application, various references describe the state of the art to which this invention pertains. The disclosures of these references are hereby incorporated by reference into the present disclosure.

1. A method for predicting the responsiveness of a patient to an anti-TNFα antibody treatment, said method comprising the step of measuring the level of IL-15 in a biological sample obtained from said patient, wherein a high level of IL-15 is predictive of a response to an anti-TNFα antibody treatment.

2. The method of claim 1, wherein said patient is suffering from an inflammatory disease.

3. The method of claim 2, wherein said patient is suffering from a disease selected from the group consisting of inflammatory bowel diseases, rheumatoid arthritis, ankylosing spondylitis and psoriasis.

4. The method of claim 3, wherein said patient is suffering from an inflammatory bowel disease.

5. The method of claim 4, wherein said patient is suffering from Crohn's disease.

6. The method according to claim 1, wherein said biological sample is a blood sample.

7. The method according to claim 6, wherein said biological sample is a serum sample.

8. The method according to claim 1, wherein said anti-TNFα antibody is selected from the group consisting of infliximab, adalimumab and Certolizumab pegol.

9. The method according to claim 8, wherein said anti-TNFα antibody is infliximab.

10. A method for treating a patient suffering from an inflammatory disease, comprising administering an effective amount of an anti-TNFα antibody to said patient, wherein said patient has a high level of IL-15.

11. The method according to claim 10, wherein said patient is suffering from a disease selected from the group consisting of inflammatory bowel diseases, rheumatoid arthritis, ankylosing spondylitis and psoriasis.

12. The method according to claim 10, wherein said patient is suffering from an inflammatory bowel disease.

13. The method according to claim 10, wherein said patient is suffering from Crohn's disease.

14. The method according to claim 10, wherein said anti-TNFα antibody is selected from the group consisting of infliximab, adalimumab and Certolizumab pegol.

15. The method according to claim 14, wherein said anti-TNFα antibody is infliximab.
